SEQUENCE LISTING (39 sequences)

SEQ ID NO: 1 - protein, 166 aa, Artificial Sequence
  Synthetic consensus sequence
  VAR_SEQ 7..7: X = Phe or Tyr
  VAR_SEQ 8..8: X = Ser or Thr
  VAR_SEQ 9..9: X = Ala or Val
  VAR_SEQ 10..10: X = Val or Ile
  VAR_SEQ 11..11: X = Ser, Ala or Asn
  VAR_SEQ 12..12: X = Glu or no amino acid
  VAR_SEQ 13..13: X = Asn or no amino acid
  VAR_SEQ 14..14: X = Glu or no amino acid
  VAR_SEQ 15..15: X = Met, Ser or Ala
  VAR_SEQ 16..16: X = Thr or Glu
  VAR_SEQ 17..17: X = Tyr or Val
  VAR_SEQ 19..19: X = Met, Val, Asn or Tyr
  VAR_SEQ 22..22: X = Ser or Ala
  VAR_SEQ 23..23: X = Asn, Asp or no amino acid
  VAR_SEQ 24..24: X = Phe, Leu or no amino acid
  VAR_SEQ 25..25: Xaa can be any naturally occurring amino acid
  VAR_SEQ 26..26: X = Asp, Thr or no amino acid
  VAR_SEQ 27..27: X = Ala, Ile or no amino acid
  VAR_SEQ 28..28: X = Val, Leu or Ile
  VAR_SEQ 29..29: X = Ala, Val or Gly
  VAR_SEQ 30..30: X = Ala or Asp
  VAR_SEQ 31..31: X = Thyr, Val or Thr
  VAR_SEQ 32..32: X = Leu or Thr
  VAR_SEQ 33..33: X = Ala or Pro
  VAR_SEQ 34..34: X = Pro or Ser
  VAR_SEQ 35..35: X = Ala or Ile
  VAR_SEQ 36..36: X = Met or Trp
  VAR_SEQ 37..37: X = Gly, Thr or Asn
  VAR_SEQ 38..38: X = Ala, Thr or Leu
  VAR_SEQ 39..39: X = Ala, Asp, or Glu
  VAR_SEQ 40..40: X = Gln, Asn, or Ser
  VAR_SEQ 41..41: X = Trp or Val
  VAR_SEQ 42..42: X = Asn, Thr, Lys or Arg
  VAR_SEQ 43..43: X = Asn, Thr, Lys, or Arg
  VAR_SEQ 44..44: X = Phe or Val
  VAR_SEQ 45..45: X = His, Ser or Asn
  VAR_SEQ 46..46: X = Lys, Ala, Thr or Ser
  VAR_SEQ 48..48: X = Leu, Val or Ile
  VAR_SEQ 49..49: X = Ile or Val
  VAR_SEQ 50..50: X = Thr or Lys
  VAR_SEQ 51..51: X = Ile or Leu
  VAR_SEQ 52..52: X = Val or Ile
  VAR_SEQ 53..53: X = Gly or Ser
  VAR_SEQ 55..55: X = Lys, Ser or Thr
  VAR_SEQ 56..56: X = Tyr or Phe
  VAR_SEQ 57..57: X = Val, Leu, Phe or Tyr
  VAR_SEQ 58..58: X = Gln, ala, Lys or Ser
  VAR_SEQ 59..59: X = Gly, Lys; Ser or His
  VAR_SEQ 60..60: X = Phe, Tyr, Thr or Leu
  VAR_SEQ 61..61: X = Leu, Thr, Ile or Val
  VAR_SEQ 62..62: X = Asp, Asn, Ser or Lys
  VAR_SEQ 63..63: X = Asn, Asp, Arg or Ala
  VAR_SEQ 64..64: X = Thr or Val
  VAR_SEQ 65..65: X = Gly or Ala
  VAR_SEQ 66..66: X = Gly or Ala
  VAR_SEQ 67..67: X = Ala, Gln, Val or Thr
  VAR_SEQ 68..68: X = Val, Met or Leu
  VAR_SEQ 70..70: X = Ser, Asp or Gly
  VAR_SEQ 72..72: X = Thr, Asn or Ser
  VAR_SEQ 73..73: X = Trp, Tyr or no amino acid
  VAR_SEQ 74..74: X = Thr, Val or Gly
  VAR_SEQ 75..75: X = Pro, Thr, Lys or Ser
  VAR_SEQ 76..76: X = Gly, Trp or Asp
  VAR_SEQ 77..77: X = Asp, Lys or Gly
  VAR_SEQ 78..78: X = Gly, Glu, Asp, Val or no amino acid
  VAR_SEQ 79..79: X = Leu, Val, Lys or Thr
  VAR_SEQ 80..80: X = Thr, Ile, Gly or Leu
  VAR_SEQ 81..81: X = Gly, Asn or Phe
  VAR_SEQ 82..82: X = Phe, Tyr, Ile or Gly
  VAR_SEQ 83..83: X = Gly, Ser, Glu or Asp
  VAR_SEQ 84..84: X = Gly or no amino acid
  VAR_SEQ 85..85: X = Gln or no amino acid
  VAR_SEQ 86..86: X = Phe or no amino acid
  VAR_SEQ 87..87: X = Ser, Val or no amino acid
  VAR_SEQ 88..88: X = Lys, Thr or no amino acid
  VAR_SEQ 89..89: X = Ile, Asn or no amino acid
  VAR_SEQ 90..90: X = Trp, Leu or no amino acid
  VAR_SEQ 91..91: X = Lys, Asp, Glu or Asn
  VAR_SEQ 92..92: X = Asp, Lys, Asn or Gly
  VAR_SEQ 93..93: X = Asn, Ala, Leu, Ser or Thr
  VAR_SEQ 94..94: X = Tyr, Phe or Ile
  VAR_SEQ 95..95: X = Thr, Asn, Gly or Ser
  VAR_SEQ 96..96: X = Asp, Thr, Gln or no amino acid
  VAR_SEQ 97..97: X = Asn or no amino acid
  VAR_SEQ 98..98: X = Val or no amino acid
  VAR_SEQ 99..99: Xaa can be any naturally occurring amino acid
  VAR_SEQ 100..100: X = Gly or Asp
  VAR_SEQ 101..101: X = Glu, Tyr, Asn or Leu
  VAR_SEQ 102..102: X = Ser, Gly, Pro, Trp or Tyr
  VAR_SEQ 103..103: X = Thr, Ile or Asn
  VAR_SEQ 104..104: X = Gly, Phe, Glu, Asp or Val
  VAR_SEQ 105..105: X = Ala, Gly, Lys, Asp or Asn
  VAR_SEQ 106..106: X = Gln, Gly, Asn, His or Arg
  VAR_SEQ 107..107: X = Lys, Asn, Thr or Leu
  VAR_SEQ 108..108: X = Phe, Trp, Thr or Pro
  VAR_SEQ 109..109: X = Gly or Arg
  VAR_SEQ 110..110: X = Tyr, Phe, Asp, Thr or Gly
  VAR_SEQ 111..111: X = Gly, Tyr, Asp, Glu or no amino acid
  VAR_SEQ 112..112: X = Ala or Asp
  VAR_SEQ 113..113: X = Leu, Val, Met or Gln
  VAR_SEQ 114..114: X = Gly, Asn or Thr
  VAR_SEQ 115..115: X = Val, Ala or Phe
  VAR_SEQ 116..116: X = Val, Leu or Gly
  VAR_SEQ 117..117: X = Asn or no amino acid
  VAR_SEQ 118..118: X = Ser, Ala, Lys or no amino acid
  VAR_SEQ 119..119: X = Ile, Gly, Val, or no amino acid
  VAR_SEQ 120..120: X = Leu, Met or no amino acid
  VAR_SEQ 121..121: X = Asn, Gln, Thr or no amino acid
  VAR_SEQ 122..122: X = Val, Ile, Thr or no amino acid
  VAR_SEQ 123..123: X = Ala, Leu or no amino acid
  VAR_SEQ 125..125: X = As, Gly, Ile, Met or Leu
  VAR_SEQ 126..126: X = Leu or Ala
  VAR_SEQ 127..127: X = Ala or Ser
  VAR_SEQ 128..128: X = Ala or Val
  VAR_SEQ 129..129: X = Ile, Val or Gly
  VAR_SEQ 131..131: X = Asn or Thr
  VAR_SEQ 134..134: X = Phe, ser, Val, Thr or Met
  VAR_SEQ 135..135: X = Gly, Ser, Ala, Thr or Lys
  VAR_SEQ 136..136: X = Thr, Ser, Pro or Asp
  VAR_SEQ 137..137: X = Ala, Ile or Thr
  VAR_SEQ 138..138: X = Lys, Gly, or Ala
  VAR_SEQ 139..139: X = Asn, Leu or Val
  VAR_SEQ 140..140: X = Ile, Glu, Thr, Gly or Leu
  VAR_SEQ 141..141: X = Val, Thr or Phe
  VAR_SEQ 142..142: X = Gly, Lys, Asn or Ala
  VAR_SEQ 143..143: X = Glu, Ser, Asp or Lys
  VAR_SEQ 144..144: X = Gly, Thr or Lys
  VAR_SEQ 145..145: X = Val, Thr or Glu
  VAR_SEQ 146..146: X = Tyr, Leu, Val or Thr
  VAR_SEQ 147..147: X = Lys, Pro, Gly or Asn
  VAR_SEQ 148..148: X = Ala, Thr, Phe, Ile or Ser
  VAR_SEQ 149..149: X = Leu, Thr, Asn or no amino acid
  VAR_SEQ 150..150: X = Val, Gly or no amino acid
  VAR_SEQ 151..151: X = Lys, Gln or no amino acid
  VAR_SEQ 152..152: X = Leu, Val or no amino acid
  VAR_SEQ 153..153: X = Trp or no amino acid
  VAR_SEQ 154..154: X = Gly or no amino acid
  VAR_SEQ 155..155: X = Asp or no amino acid
  VAR_SEQ 156..156: X = Leu or no amino acid
  VAR_SEQ 157..157: X = Pro or no amino acid
  VAR_SEQ 158..158: X = Asn or no amino acid
  VAR_SEQ 159..159: X = Asn or no amino acid
  VAR_SEQ 160..160: X = Gly or no amino acid
  VAR_SEQ 161..161: X = Gly or no amino acid
  VAR_SEQ 162..162: X = Ser or no amino acid
  VAR_SEQ 163..163: X = Gly or no amino acid
  VAR_SEQ 164..164: X = Trp or no amino acid
  VAR_SEQ 165..165: X = Val or no amino acid
  VAR_SEQ 166..166: X = Gly or no amino acid
MMMPANXXXXXXXXXXXVXGGXXXXXXXXXXXXXXXXXXXXXXXXXNXXXXXXNXXXXXXXXXXXXXXFXGXXXXXXXXXXXXXXXXXXXXXXXXXXXXXXXXXXXXXXXXXXXXXXXXXXXXGXXXXXYXLGXXXXXXXXXXXXXXXXXXXXXXXXXXXXXXXXX

SEQ ID NO: 2 - protein, 135 aa, Faecalibacterium prausnitzii
MMMPANYSVIAENEMTYVNGGANFIDAIGAVTAPIWTLDNVKTFNTNIVTLVGNTFLQSTINRTIGVLFSGNTTWKEVGNIGKNLFGTNVKGNPIEKNNFGDYAMNALGIAAAVYNLGVAPTKNTVKETEVKFTV

SEQ ID NO: 3 - protein, 143 aa, Faecalibacterium prausnitzii
MMMPANFSAVSENEMTYVMGGSVADYLAPAMGAAQWQNFHKNLITIVGNKYVQGFLDNTVGAVFSGTWTPGDGLTGFGGQFSKIWKDNYTDNVTGESTGAQKFGYGALGVVNSILNVAGNLAAIYNLGFGTAKNIVGEGVYKA

SEQ ID NO: 4 - protein, 143 aa, Faecalibacterium prausnitzii
MMMPANFSAVSENEMTYVMGGSVADYLAPAMGAAQWQNFHKNLITIVGNKYVQGFLDNTVGAMFSGTWTPGDGLTGFGGQFSTIWKKNYTDNVTDESTGAQKFGYGALGVVNSILNVAGNLAAIYNLGFGTAKNIVGEGVYKA

SEQ ID NO: 5 - protein, 136 aa, Faecalibacterium prausnitzii
MMMPANFSAVAENEMTYVVGGSLVDVLAPAMTTANWQNVSANVIKIVGNSFLAKYTNDVLAQLFDGNYVPGDVIGYSVKNLDKAYNKGYGTFGGNWGFAVGALNAGMQILGGLSAIYTLGSSSIGLETKSGTLPTL

SEQ ID NO: 6 - protein, 142 aa, Faecalibacterium prausnitzii
MMMPANFTAVNSEVVYGGADLFTILADTTAPIWNAANVKKFNTNLITLISNSFFKKTVSNTLGVMFGGNWGKDGDKIFGEEGSINQNVFGLWNDDHTTRTDDMTFGNKVMQVLGMAAVGYTLGTTDAKVGFNDGVYGINGKL

SEQ ID NO: 7 - protein, 147 aa, Faecalibacterium prausnitzii
MMMPANFSAVNAEVVYGGAVADYLPSAWTAESVKRFNSNIITLVSNSFTSHLLKATLGTMFSGSWGSDGVTLFGDNGTFSGLYNVNRLPGGEAQTFGNKIMTTLGLASVVYTLGMKDAAVLTAKKVTNSNGQVWGDLPNNGGSGWVG

SEQ ID NO: 8 - protein, 20 aa, Artificial Sequence
  Synthetic p49-70 portion of SEQ ID NO2
VTLVGNTFLQSTINRTIGVL

SEQ ID NO: 9 - protein, 43 aa, Artificial Sequence
  Synthetic p35-77 portion of SEQ ID NO2
IWTLDNVKTFNTNIVTLVGNTFLQSTINRTIGVLFSGNTTWKE

SEQ ID NO: 10 - protein, 20 aa, Artificial Sequence
  Synthetic SL3/3 fragment
ITIVGNKYVQGFLDNTVGAV

SEQ ID NO: 11 - protein, 20 aa, Artificial Sequence
  Synthetic M21/2 fragment
ITIVGNKYVQGFLDNTVGAM

SEQ ID NO: 12 - protein, 20 aa, Artificial Sequence
  Synthetic KLE1255 fragment
IKIVGNSFLAKYTNDVLAQL

SEQ ID NO: 13 - protein, 20 aa, Artificial Sequence
  Synthetic L2-61 fragment
ITLISNSFFKKTVSNTLGVM

SEQ ID NO: 14 - protein, 20 aa, Artificial Sequence
  Synthetic L2-62 fragment
ITLVSNSFTSHLLKATLGTM

SEQ ID NO: 15 - protein, 20 aa, Artificial Sequence
  Synthetic CAG821 fragment
VTIIGNSYVSKLVGATLGVM

SEQ ID NO: 16 - protein, 20 aa, Artificial Sequence
  Synthetic CAG822 fragment
ITIIGNSYVSKVLGATLGVM

SEQ ID NO: 17 - protein, 18 aa, Artificial Sequence
  Synthetic CNCM-4573 fragment
IVGNKYVQGFLDNTVGAV

SEQ ID NO: 18 - protein, 43 aa, Artificial Sequence
  Synthetic SL3/3 fragment
AMGAAQWQNFHKNLITIVGNKYVQGFLDNTVGAVFSGTWTPGD

SEQ ID NO: 19 - protein, 43 aa, Artificial Sequence
  Synthetic M21/2 fragment
AMGAAQWQNFHKNLITIVGNKYVQGFLDNTVGAMFSGTWTPGD

SEQ ID NO: 20 - protein, 43 aa, Artificial Sequence
  Synthetic KLE1255 fragment
AMTTANWQNVSANVIKIVGNSFLAKYTNDVLAQLFDGNYVPGD

SEQ ID NO: 21 - protein, 43 aa, Artificial Sequence
  Synthetic L2-61 fragment
IWNAANVKKFNTNLITLISNSFFKKTVSNTLGVMFGGNWGKDG

SEQ ID NO: 22 - protein, 43 aa, Artificial Sequence
  Synthetic L2-62 fragment
AWTAESVKRFNSNIITLVSNSFTSHLLKATLGTMFSGSWGSDG

SEQ ID NO: 23 - protein, 43 aa, Artificial Sequence
  Synthetic CAG821 fragment
IWTTANVKTFNTNLVTIIGNSYVSKLVGATLGVMFGGNWGGDG

SEQ ID NO: 24 - protein, 43 aa, Artificial Sequence
  Synthetic CAG822 fragment
VWGAANVKTFNTNLITIIGNSYVSKVLGATLGVMFSGAWGTKD

SEQ ID NO: 25 - protein, 43 aa, Artificial Sequence
  Synthetic CNCM-4573 fragment
AMGAAQWQNFHKNLVTIVGNKYVQGFLDNTVGAVFSGTWTPGV

SEQ ID NO: 26 - protein, 43 aa, Faecalibacterium prausnitzii
FSGNTTWKEVGNIGKNLFGTNVKGNPIEKNNFGDYAMNALGIA

SEQ ID NO: 27 - protein, 16 aa, Faecalibacterium prausnitzii
GNTFLQSTINRTIGVL

SEQ ID NO: 28 - protein, 17 aa, Faecalibacterium prausnitzii
VGNTFLQSTINRTIGVL

SEQ ID NO: 29 - protein, 18 aa, Faecalibacterium prausnitzii
LVGNTFLQSTINRTIGVL

SEQ ID NO: 30 - protein, 19 aa, Faecalibacterium prausnitzii
TLVGNTFLQSTINRTIGVL

SEQ ID NO: 31 - protein, 20 aa, Faecalibacterium prausnitzii
VTLVGNTFLQSTINRTIGVL

SEQ ID NO: 32 - protein, 42 aa, Artificial Sequence
  Synthetic Fragment
IWTLDNVKTFNTNIVTLVGNTFLQSTINRTIGVLFSGNTTWK

SEQ ID NO: 33 - protein, 99 aa, Artificial Sequence
  Synthetic MUT3
MMMPANYSVIAENEMTYVNGGANFIDAIGAVTAPIWTLDNVKTFNTNIVTLVGNTFLQSTINRTIGVLFSGNTTWKEVGNIGKNLFGTNVKGNPIEKNN

SEQ ID NO: 34 - protein, 113 aa, Artificial Sequence
  Synthetic MUT1
NFIDAIGAVTAPIWTLDNVKTFNTNIVTLVGNTFLQSTINRTIGVLFSGNTTWKEVGNIGKNLFGTNVKGNPIEKNNFGDYAMNALGIAAAVYNLGVAPTKNTVKETEVKFTV

SEQ ID NO: 35 - protein, 15 aa, Faecalibacterium prausnitzii
NTFLQSTINRTIGVL

SEQ ID NO: 36 - protein, 24 aa, Faecalibacterium prausnitzii
AAVYNLGVAPTKNTVKETEVKFTV

SEQ ID NO: 37 - protein, 47 aa, Faecalibacterium prausnitzii
NYSVIAENEMTYVNGGANFIDAIGAVTAPIWTLDNVKTFNTNIVTLV

SEQ ID NO: 38 - protein, 17 aa, Faecalibacterium prausnitzii
KGNTFLQSTINRTIGVL

SEQ ID NO: 39 - protein, 46 aa, Faecalibacterium prausnitzii
VKGNPIEKNNFGDYAMNALGIAAAVYNLGVAPTKNTVKETEVKFTV

(19) United States
(12) Patent Application Publication
(10) Pub. No.: US 2021/0393737 A1
(43) Pub. Date: Dec. 23, 2021

(54) USE OF MAM POLYPEPTIDES FOR THE TREATMENT OF OBESITY AND OBESITY-RELATED DISORDERS

(71) Applicants: INSERM (INSTITUT NATIONAL DE LA SANTÉ ET DE LA RECHERCHE MÉDICALE), Paris (FR); INSTITUT NATIONAL DE RECHERCHE POUR L'AGRICULTURE, L'ALIMENTATION ET L'ENVIRONNENT, Paris (FR); UNIVERSITÉ PAUL SABATIER TOULOUSE III, TOULOUSE (FR)

(72) Inventors: Christophe HEYMES, St Orens de Gameville (FR); Rémy BURCELIN, Auzeville-Tolosane (FR); Jean-Marc CHATEL, MEUDON (FR); Philippe LANGELLA, Vélizy-Villacoublay (FR)

(21) Appl. No.: 17/279,701

(22) PCT Filed: Sep. 24, 2019
(86) PCT No.: PCT/EP2019/075755

§ 371 Date: Mar. 25, 2021

(30) Foreign Application Priority Data
Sep. 25, 2018 (EP) 18306245.4

(51) Int. Cl. A61K 38/16; A61K 35/744; A61P 3/04

(52) U.S. Cl. CPC A61K 38/164; A61P 3/04; A61K 35/744

(57) ABSTRACT

The development of obesity is still increasing worldwide. Likewise, metabolic syndrome, which is a collection of obesity-associated disorders, is associated with development of cardiovascular diseases, insulin resistance, hepatic steatosis, certain types of cancer and type 2 diabetes. Recently, seven peptides were isolated from F.prausnitzii and were shown as deriving from a single Microbial Anti-inflammatory Molecule (MAM). The inventors shows that L-MAM treatment prevented animals from development of High-Fat Diet-induced obesity. L-MAM treated mice significantly gained less weight throughout the gavage as compared to HFD-fed control experiment. The difference in body weight between L-MAM-treated animals on a HFD and the remaining animals is largely due to a significant reduction in the percentage of fat mass and a significant increase in the percentage of lean mass. Oral glucose tolerance testing (OGTT) revealed that chronic administration of L-MAM increased glucose tolerance. Finally, all adipose tissues weights were lower in L-MAM-treated mice. Accordingly, the present invention relates to the use of MAM polypeptides for the treatment of obesity and obesity-related disorders.

### FIELD OF THE INVENTION

The present invention relates to the use of MAM polypeptides for the treatment of obesity and obesity-related disorders.

### BACKGROUND OF THE INVENTION

Excess fat mass leads to overweight and obesity, which have deleterious health consequences. The development of obesity is still increasing worldwide. Likewise, metabolic syndrome, which is a collection of obesity-associated disorders, is associated with development of cardiovascular diseases, insulin resistance, hepatic steatosis, certain types of cancer and type 2 diabetes. Resistance of peripheral tissues (liver, muscle, adipose tissue) to insulin action is indeed a key event in diabetes onset, so that therapeutic strategies aiming at restoring insulin sensitivity are highly relevant. However, they essentially remain unsatisfactory. For instance, thiazolidinediones, by binding peroxisome proliferator activated receptor gamma, increase insulin sensitivity, but such treatments are associated to important side effects. There is therefore still an important need to understand obesity-promoting mechanisms to identify therapeutic targets. Recently, seven peptides were isolated from identified in the F. prausnitzii and were shown as deriving from a single Microbial Anti-inflammatory Molecule (MAM) (Quevrain, E., et al. “Identification of an anti-inflammatory protein from Faecalibacterium prausnitzii, a commensal bacterium deficient in Crohn's disease.” Gut, 2015). The authors demonstrated that the use of a food-grade bacterium, Lactococcus lactis, delivering a plasmid carrying the cDNA of MAM was able to alleviate colitis in mice. However, the role of these peptides in obesity was not suspected in the prior art.

### SUMMARY OF THE INVENTION

The present invention relates to the use of MAM polypeptides for the treatment of obesity and obesity-related disorders. In particular, the present invention is defined by the claims.

### DETAILED DESCRIPTION OF THE INVENTION

The present text describes a method of treating obesity in a subject in need thereof comprising administering to the subject a therapeutically effective amount of:
a polypeptide having the amino acid sequence as set forth in SEQ ID NO:1 or fragment thereof,
a nucleic acid molecule encoding for a polypeptide having the amino acid sequence as set forth in SEQ ID NO:1 or a fragment thereof,
a vector comprising a nucleic acid molecule encoding for a polypeptide having the amino acid sequence as set forth in SEQ ID NO:1 or a fragment thereof,
a host cell transformed with a nucleic acid molecule encoding for a polypeptide having the amino acid sequence as set forth in SEQ ID NO:1 or a fragment thereof.

In particular, the first object of the present invention relates to a method of treating obesity or an obesity-related disease in a subject in need thereof comprising administering to the subject a therapeutically effective amount of:
a polypeptide having the amino acid sequence as set forth in SEQ ID NO:1 or a fragment thereof,
a nucleic acid molecule encoding for a polypeptide having the amino acid sequence as set forth in SEQ ID NO:1 or a fragment thereof,
a vector comprising a nucleic acid molecule encoding for a polypeptide having the amino acid sequence as set forth in SEQ ID NO:1 or a fragment thereof, and/or
a host cell transformed with a nucleic acid molecule encoding for a polypeptide having the amino acid sequence as set forth in SEQ ID NO:1 or a fragment thereof,
   wherein the obesity-related disease is selected from the group consisting of bulimia, diabetes, hypertension, elevated plasma insulin concentrations and insulin resistance, dyslipidemia, hyperlipidemia, breast, prostate, endometrial cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack, and osteoarthritis,
   or from the group consisting of metabolic syndrome, also known as syndrome X, insulin resistance syndrome, type II diabetes, impaired fasting glucose, impaired glucose tolerance, hypercholesterolemia, hyperuricaemia, and left ventricular hypertrophy, or is a non-alcoholic fatty liver disease,
   and is in particular a non-alcoholic fatty liver disease.

In a particular embodiment, the obesity-related disease is a non-alcoholic fatty liver disease that is nonalcoholic steatohepatitis (NASH).

In another particular embodiment, the obesity-related disease is insulin resistance.

As used herein, the term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m2 to less than 25 kg/m².

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The method of the present invention is particularly suitable for the treatment (e.g. prophylactic treatment) of obesity related disorders.

As used herein, the term “obesity-related diseases” encompasses disorders that are associated with, caused by, or result from obesity. Examples of obesity-related disorders include overeating and bulimia, diabetes, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack, and osteoarthritis. Other examples include pathological conditions showing reduced metabolic activity or a decrease in resting energy expenditure as a percentage of total fat-free mass. Further examples of obesity-related disorders include metabolic syndrome, also known as syndrome X, insulin resistance syndrome, type II diabetes, impaired fasting glucose, impaired glucose tolerance, inflammation, such as systemic inflammation of the vasculature, atherosclerosis, hypercholesterolemia, hyperuricaemia, as well as secondary outcomes of obesity such as left ventricular hypertrophy. Obesity-related disorders also include the liver abnormalities associated with obesity such as non-alcoholic fatty liver disease (NAFLD) a rising cause of cirrhosis associated to obesity and metabolic syndrome. Indeed, NAFLD can present as simple steatosis or evolve towards inflammation and steatohepatitis (NASH), with a 20% risk of cirrhosis after 20 years. “Dyslipidemia” is a major risk factor for coronary heart disease (CHD). Low plasma levels of high density lipoprotein (HDL) cholesterol with either normal or elevated levels of low density (LDL) cholesterol is a significant risk factor for developing atherosclerosis and associated coronary artery disease in humans. Dyslipidemia is often associated with obesity.

According to the invention, obesity-related diseases are selected from the group consisting of bulimia, diabetes, hypertension, elevated plasma insulin concentrations and insulin resistance, dyslipidemia, hyperlipidemia, breast, prostate, endometrial cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack, osteoarthritis, metabolic syndrome, also known as syndrome X, insulin resistance syndrome, type II diabetes, impaired fasting glucose, impaired glucose tolerance, hypercholesterolemia, hyperuricaemia, and left ventricular hypertrophy, or are non-alcoholic fatty liver diseases, and can in particular be a non-alcoholic fatty liver disease.

In particular, an obesity-related disease according to the invention is a non-inflammatory obesity-related disease.

In some embodiments, the method of the present invention is particularly suitable for the treatment of insulin resistance. As used herein, the term “insulin resistance” has its common meaning in the art. Insulin resistance is a physiological condition where the natural hormone insulin becomes less effective at lowering blood sugars. The resulting increase in blood glucose may raise levels outside the normal range and cause adverse health effects such as metabolic syndrome, dyslipidemia and subsequently type 2 diabetes mellitus. The method of the present invention is thus particularly suitable for the treatment of type 2 diabetes. As used herein, the term “type 2 diabetes” or “non-insulin dependent diabetes mellitus (NIDDM)” has its general meaning in the art. Type 2 diabetes often occurs when levels of insulin are normal or even elevated and appears to result from the inability of tissues to respond appropriately to insulin. Most of the type 2 diabetics are obese.

In some embodiments, the method of the present invention comprises administering to the subject a therapeutically effective amount of:
a polypeptide having an amino acid sequence selected in the group comprising SEQ ID NO:2, SEQ ID NO:3, SEQ ID NO:4, SEQ ID NO:5, SEQ ID NO:6 and SEQ ID NO:7, or a fragment thereof,
a nucleic acid molecule encoding for a polypeptide having an amino acid sequence selected in the group comprising SEQ ID NO:2, SEQ ID NO:3, SEQ ID NO:4, SEQ ID NO:5, SEQ ID NO:6 and SEQ ID NO:7, or a fragment thereof,
a vector comprising a nucleic acid molecule encoding for a polypeptide having an amino acid sequence selected in the group comprising SEQ ID NO:2, SEQ ID NO:3, SEQ ID NO:4, SEQ ID NO:5, SEQ ID NO:6 and SEQ ID NO:7, or a fragment thereof,
a host cell transformed with a nucleic acid molecule encoding for a polypeptide having an amino acid sequence selected in the group comprising SEQ ID NO:2, SEQ ID NO:3, SEQ ID NO:4, SEQ ID NO:5, SEQ ID NO:6 and SEQ ID NO:7 a fragment thereof.

As used herein, the term “amino acid” refers to natural or unnatural amino acids in their D and L stereoisomers for chiral amino acids. It is understood to refer to both amino acids and the corresponding amino acid residues, such as are present, for example, in peptidyl structure. Natural and unnatural amino acids are well known in the art. Common natural amino acids include, without limitation, alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

As used herein, the term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications that include but are not limited to phosphorylation, acetylation, glycosylation and the like. By an “isolated” polypeptide, it is intended that the polypeptide is not present within a living organism, e.g. within human body. However, the isolated polypeptide may be part of a composition or a kit. The isolated polypeptide is preferably purified and or recombinant.

As use herein the term “fragment” denotes a polypeptide consisting 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; consecutive amino acid residues in SEQ ID NO:1, SEQ ID NO:2, SEQ ID NO:3, SEQ ID NO:4, SEQ ID NO:5, SEQ ID NO:6 or SEQ ID NO:7.

In a particular embodiment, a fragment according denotes a polypeptide having an amino acid sequence selected from the group consisting of SEQ ID NO: 8-39.

In particular, a fragment according to the invention demotes a polypeptide having an amino acid sequence selected in the group consisting of SEQ ID NO: 8, SEQ ID NO: 26 and SEQ ID NO: 36.

The polypeptides of the present invention are produced by any technique known per se in the art, such as, without limitation, any chemical, biological, genetic or enzymatic technique, either alone or in combination. For instance, knowing the amino acid sequence of the desired sequence, one skilled in the art can readily produce said polypeptides, by standard techniques for production of amino acid sequences. For instance, they can be synthesized using well-known solid phase method, preferably using a commercially available peptide synthesis apparatus (such as that made by Applied Biosystems, Foster City, Calif.) and following the manufacturer's instructions. Alternatively, the polypeptides of the present invention can be synthesized by recombinant DNA techniques as is now well-known in the art. For example, these fragments can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques. Polypeptides or fusion proteins of the invention can be used in an isolated (e.g., purified) form or contained in a vector, such as a membrane or lipid vesicle (e.g. a liposome).

As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

As used herein, the term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication-defective retroviruses, adenoviruses and adeno-associated viruses), which serve equivalent functions. Such vectors may comprise regulatory elements, such as a promoter, enhancer, terminator and the like, to cause or direct expression of said antibody upon administration to a subject.

As used herein, the term “host cell” is intended to refer to a cell (prokaryotic cell or eukaryotic cell) into which a recombinant expression vector has been introduced in order to express a polypeptide of interest. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein. As used herein, the term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

In some embodiments the host cell is a bacterium which has been genetically engineered for expressing the polypeptide of the present invention. Methods for transforming bacterial cell with extracellular nucleic acids are well known in the art.

In some embodiments, the host cell is a probiotic bacterial strain. As used herein the term “probiotic” is meant to designate live microorganisms which, they are integrated in a sufficient amount, exert a positive effect on health, comfort and wellness beyond traditional nutritional effects. Probiotic microorganisms have been defined as “Live microorganisms which when administered in adequate amounts confer a health benefit on the host” (FAO/WHO 2001). As used herein the expression “probiotic bacterial strain” denotes a bacterial strain that has a beneficial effect on the health and well-being of the host. Non limiting examples of probiotics include: Bifidobacterium, Lactobacillus, Lactococcus, Enterococcus, Streptococcus, Kluyveromyces, Saccharomyces, Candida, in particular selected from the group consisting of Bifidobacterium longum, Bifidobacterium lactis, Bifidobacterium a ni ma Us, Bifidobacterium breve, Bidobacterium infantis, Bidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifdobacterium longum NCC3001 (ATCC BAA-999), Bifidobacterium longum NCC2705 (CNCM 1-2618), Bifidobacterium longum NCC490 (CNCM 1-2170), Bifdobacterium lactis NCC2818 (CNCM I-3446), Bifdobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), and Enterococcus faecium SF 68 (NCC2768; NCIMB10415).

In a particular embodiment, the probiotic bacterial strain is Lactococcus lactis.

In some embodiments, the probiotic bacterial strain of the present invention is a viable probiotic bacterial strain. The expression “viable probiotic bacterial strain” means a microorganism which is metabolically active and that is able to colonize the gastro-intestinal tract of the subject.

In some embodiments, the probiotic bacterial strain of the present invention is a non-viable probiotic bacterial strain consisting of a mixture of bacterial fragments. In some embodiments, the mixture of bacterial fragments of the present invention consists of proteins from the bacterial strain.

In some embodiments, the probiotic bacterial stain of the present invention is selected from food grade bacteria. “Food grade bacteria” means bacteria that are used and generally regarded as safe for use in food.

Typically, the probiotic bacterial strain of the present invention is produced with any appropriate culture medium well known in the art. Various fermentation media are suitable according to the invention, such as (but not limited to) e.g. firstly an industrial medium, in which the strain(s) is/are grown, and that is used as is or after concentration (e.g. drying) or after addition to another food base or product. Alternatively, bacterial cells, or bacterial cells with medium (e.g. the fermentation broth), or fractions of such cell comprising medium (i.e. medium with said bacterial strain/s) may be used. The cells or the cell comprising medium comprise live or viable bacterial cells and/or dead or non-viable bacterial cells of the strain(s). The medium may thus be treated by, but not limited to, heating or sonication. Also lyophilized, or frozen, bacteria and/or cell-free media (which may be concentrated) are encompassed in the methods for preparing the probiotic bacterial strain of the present invention.

Typically, the probiotic bacterial strain of the present invention is administered to the subject by ingestion (i.e. oral route).

In some embodiments, the probiotic bacterial strain of the present invention is encapsulated in order to be protected against the stomach. Accordingly, in some embodiments the probiotic bacterial strain of the present invention is formulated in compositions in an encapsulated form so as significantly to improve their survival time. In such a case, the presence of a capsule may in particular delay or prevent the degradation of the microorganism in the gastrointestinal tract. It will be appreciated that the compositions of the present embodiments can be encapsulated into an enterically-coated, time-released capsule or tablet. The enteric coating allows the capsule/tablet to remain intact (i.e., undissolved) as it passes through the gastrointestinal tract, until such time as it reaches the small intestine. Methods of encapsulating live bacterial cells are well known in the art (see, e.g., U.S. patents to General Mills Inc. such as U.S. Pat. No. 6,723,358). For example, micro-encapsulation with alginate and Hi-Maize™ starch followed by freeze-drying has been proved successful in prolonging shelf-life of bacterial cells in dairy products [see, e.g., Kailasapathy et al. Curr Issues Intest Microbiol. 2002 September; 3(2):39-48]. Alternatively encapsulation can be done with glucomannane fibers such as those extracted from Amorphophallus konjac. Alternatively, entrapment of viable probiotic in sesame oil emulsions may also be used [see, e.g., Hou et al. J. Dairy Sci. 86:424-428]. In some embodiments, agents for enteric coatings are preferably methacrylic acid-alkyl acrylate copolymers, such as Eudragit® polymers. Poly(meth)acrylates have proven particularly suitable as coating materials. EUDRAGIT® is the trade name for copolymers derived from esters of acrylic and methacrylic acid, whose properties are determined by functional groups. The individual EUDRAGIT® grades differ in their proportion of neutral, alkaline or acid groups and thus in terms of physicochemical properties. The skillful use and combination of different EUDRAGIT® polymers offers ideal solutions for controlled drug release in various pharmaceutical and technical applications. EUDRAGIT® provides functional films for sustained-release tablet and pellet coatings. The polymers are described in international pharmacopeias such as Ph. Eur., USP/NF, DMF and JPE. EUDRAGIT® polymers can provide the following possibilities for controlled drug release: gastrointestinal tract targeting (gastroresistance, release in the colon), protective coatings (taste and odor masking, protection against moisture) and delayed drug release (sustained-release formulations). EUDRAGIT® polymers are available in a wide range of different concentrations and physical forms, including aqueous solutions, aqueous dispersion, organic solutions, and solid substances. The pharmaceutical properties of EUDRAGIT® polymers are determined by the chemical properties of their functional groups. A distinction is made between:
poly(meth)acrylates, soluble in digestive fluids (by salt formation) EUDRAGIT® L (Methacrylic acid copolymer), S (Methacrylic acid copolymer), FS and E (basic butylated methacrylate copolymer) polymers with acidic or alkaline groups enable pH-dependent release of the active ingredient. Applications: from simple taste masking via resistance solely to gastric fluid, to controlled drug release in all sections of the intestine.
poly(meth)acrylates, insoluble in digestive fluids: EUDRAGIT® RL and RS (ammonio methacrylate copolymers) polymers with alkaline and EUDRAGIT® NE polymers with neutral groups enable controlled time release of the active by pH-independent swelling.
   Enteric EUDRAGIT® coatings provide protection against drug release in the stomach and enable controlled release in the intestine. The dominant criterion for release is the pH-dependent dissolution of the coating, which takes place in a certain section of the intestine (pH 5 to over 7) rather than in the stomach (pH 1-5). For these applications, anionic EUDRAGIT® grades containing carboxyl groups can be mixed with each other. This makes it possible to finely adjust the dissolution pH, and thus to define the drug release site in the intestine. EUDRAGIT® L and S grades are suitable for enteric coatings. EUDRAGIT® FS 30 D (aqueous dispersion of an anionic copolymer based on methyl acrylate, methyl methacrylate and methacrylic acid) is specifically used for controlled release in the colon.

Typically, the probiotic bacterial strain of the present invention is administered to the subject in the form of a food composition. Accordingly one further aspect of the present invention relates to a food composition comprising an amount of the probiotic bacterial strain of the present invention.

In some embodiments, the food composition that comprises the probiotic bacterial strain of the present invention is selected from complete food compositions, food supplements, nutraceutical compositions, and the like. The composition of the present invention may be used as a food ingredient and/or feed ingredient. The food ingredient may be in the form of a solution or as a solid—depending on the use and/or the mode of application and/or the mode of administration. The probiotic bacterial strain of the present invention is typically added at any time during the production process of the composition, e.g. they may be added to a food base at the beginning of the production process or they may be added to the final food product.

In a particular embodiment, the food composition that comprises the probiotic bacterial strain comprises an amount of dietary fibers.

As used herein, the term “food” refers to liquid (i.e. drink), solid or semi-solid dietetic compositions, especially total food compositions (food-replacement), which do not require additional nutrient intake or food supplement compositions. Food supplement compositions do not completely replace nutrient intake by other means. Food and food supplement compositions are for example fermented dairy products or dairy-based products, which are preferably administered or ingested orally one or more times daily. Fermented dairy products can be made directly using the bacteria according to the invention in the production process, e.g. by addition to the food base, using methods known per se. In such methods, the strain(s) of the invention may be used in addition to the micro-organism usually used, and/or may replace one or more or part of the micro-organism usually used. For example, in the preparation of fermented dairy products such as yoghurt or yoghurt-based drinks, a bacterium of the invention may be added to or used as part of a starter culture or may be suitably added during such a fermentation. Optionally the bacteria may be inactivated or killed later in the production process. Fermented dairy products include milk-based products, such as (but not limited to) deserts, yoghurt, yoghurt drinks, quark, kefir, fermented milk-based drinks, buttermilk, cheeses, dressings, low fat spreads, fresh cheese, soy-based drinks, ice cream, etc. Alternatively, food and/or food supplement compositions may be non-dairy or dairy non fermented products (e.g. strains or cell-free medium in non fermented milk or in another food medium). In some embodiments, the probiotic bacterial strain of the present invention is encapsulated and dispersed in a food (e.g. in milk) or non food medium. Non-fermented dairy products may include ice cream, nutritional bars and dressings, and the like. Non-dairy products may include powdered beverages and nutritional bars, and the like. The products may be made using known methods, such as adding an effective amount of the strain(s) and/or cell-free culture medium to a food base, such as skimmed milk or milk or a milk-based composition and fermentation as known. Other food bases to which the (compositions comprising the) bacterial cells and/or cell-free culture medium may be added are meat, meat replacers or plant bases.

The composition that comprises the probiotic bacterial strain of the present invention may be solid, semi-solid or liquid. It may be in the form of a food product or food supplement, e.g. in the form of tablets, gels, powders, capsules, drinks, bars, etc. For example the composition may be in the form of a powder packed in a sachet which can be dissolved in water, fruit juice, milk or another beverage.

As used herein the term “food ingredient” or “feed ingredient” includes a formulation which is or can be added to functional foods or foodstuffs as a nutritional supplement.

By “nutritional food” or “nutraceutical” or “functional” food, is meant a foodstuff which contains ingredients having beneficial effects for health or capable of improving physiological functions.

By “food supplement”, is meant a foodstuff having the purpose of completing normal food diet. A food supplement is a concentrated source of nutrients or other substances having a nutritional or physiological effect, when they are taken alone or as a combination in small amounts.

According to the invention, “functional food” summarizes foodstuff and corresponding products lately developed to which importance is attributed not only due to them being valuable as to nutrition and taste but due to particular ingredient substances. According to the invention, the middle- or long-term maintenance and promotion of health are of importance. In this context, non-therapeutic uses are preferred. The terms “nutriceuticals”, “foodsceuticals” and “designer foods”, which also represent embodiments of the invention, are used as synonyms, partly, however, also in a differentiated way. The preventive aspect and the promotion of health as well as the food character of the products are, however, best made clear by the term functional food. In many cases, these relate to products accumulated by assortment and selection (as is also the case in the present invention), purification, concentration, increasingly also by addition. Isolated effective substances, in particular in form of tablets or pills, are not included. Although there is no legal definition of a functional food, most of the parties with an interest in this area agree that they are foods marketed as having specific health effects beyond basic nutritional effects. Accordingly, functional foods are ordinary foods that have components or ingredients (such as those described herein) incorporated into them that impart to the food a specific functional e.g. medical or physiological benefit other than a purely nutritional effect.

In some embodiments, the drink is a functional drink or a therapeutic drink, a thirst-quencher or an ordinary drink. By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

The composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped tipping, low fat and light natural cheddar cheese, low fat Swiss style yoghurt, aerated frozen desserts, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

In some embodiments, the composition that comprises the probiotic bacterial strain of the present invention is used with yoghurt production, such as fermented yoghurt drink, yoghurt, drinking yoghurt, cheese, fermented cream, milk based desserts and others. Suitably, the composition can be further used as an ingredient in one or more of cheese applications, meat applications, or applications comprising protective cultures.

In some embodiments, the food composition that comprises the probiotic bacterial strain of the present invention is suitable for preparing meal replacement product. As used herein, the term “meal replacement product” as used herein, unless otherwise specified, includes any nutritional product containing protein, carbohydrate, lipid, vitamins and minerals, the combination of which is then suitable as a sole or primary nutrition source for a meal. Typically, the meal replacement product comprises at least one carbohydrate source, at least one lipid source and/or at least one protein source. As protein source any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey, and soy proteins are particularly preferred. The proteins may be intact or hydrolysed or a mixture of intact and hydrolysed proteins. It may be desirable to supply partially hydrolysed proteins (degree of hydrolysis between 2 and 20%), for example for animals believed to be at risk of developing cows' milk allergy. If hydrolysed proteins are required, the hydrolysis process may be carried out as desired and as is known in the art. For example, a whey protein hydrolysate may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source. If the composition includes a fat source, the fat source preferably provides 5% to 40% of the energy of the composition; for example 20% to 30% of the energy. A suitable fat profile may be obtained using a blend of canola oil, corn oil and high-oleic acid sunflower oil. The source of carbohydrates preferably provides 40% to 80% of the energy of the composition. Any suitable carbohydrate may be used, for example sucrose, lactose, glucose, fructose, corn syrup solids, maltodextrins, and mixtures thereof. Typically, substituting one daily meal by an energy restricted diet with a meal replacement contributes to the maintenance of weight after weight loss.

The food composition that comprises the probiotic bacterial strain of the present invention typically comprises carriers or vehicles. “Carriers” or “vehicles” mean materials suitable for administration and include any such material known in the art such as, for example, any liquid, gel, solvent, liquid diluent, solubilizer, or the like, which is non-toxic and which does not interact with any components of the composition in a deleterious manner. Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

In some embodiments, the food composition that comprises the probiotic bacterial strain of the present invention comprises an amount of dietary fibres. Dietary fibre passes through the small intestine undigested by enzymes and functions as a natural bulking agent and laxative. Dietary fibre may be soluble or insoluble and in general a blend of the two types is preferred. Suitable sources of dietary fibre include soy, pea, oat, pectin, guar gum, gum Arabic, fructooligosaccharides, galacto-oligosaccharides, sialyl-lactose and oligosaccharides derived from animal milks. In some embodiments, the dietary fiber is selected among mannans. Mannans (such as glucomannans and galactomannans), such as guar gum, locust bean gum, konjac, and xanthan gum, are present in some plant cell walls. The glucomannans are generally comprised of (1-4)-β-linked glucose and mannose units, while the galactomannans are generally comprised of a (1-4)-β-mannan backbone substituted with single units of (1-6)-α-galactose. Many endospermic legumes, such as guar and locust bean, contain galactomannans in the endosperm during seed development. Glucomannans have also been found as a minor component of cereal grains.

In some embodiments, the food composition that comprises the probiotic bacterial strain of the present invention contains minerals and micronutrients such as trace elements and vitamins in accordance with the recommendations of Government bodies such as the USRDA. For example, the composition may contain per daily dose one or more of the following micronutrients in the ranges given:—300 to 500 mg calcium, 50 to 100 mg magnesium, 150 to 250 mg phosphorus, 5 to 20 mg iron, 1 to 7 mg zinc, 0.1 to 0.3 mg copper, 50 to 200 μg iodine, 5 to 15 μg selenium, 1000 to 3000 μg beta carotene, 10 to 80 mg Vitamin C, 1 to 2 mg Vitamin B1, 0.5 to 1.5 mg Vitamin B6, 0.5 to 2 mg Vitamin B2, 5 to 18 mg niacin, 0.5 to 2.0 μg Vitamin B12, 100 to 800 μg folic acid, 30 to 70 μg biotin, 1 to 5 μg Vitamin D, 3 to 10 μg Vitamin E.

In some embodiments, the composition that comprises the probiotic bacterial strain of the present invention contains emulsifiers. Examples of food grade emulsifiers typically include diacetyl tartaric acid esters of mono- and di-glycerides, lecithin and mono- and di-glycerides. Similarly suitable salts and stabilisers may be included.

In some embodiments, the food composition that comprises the probiotic bacterial strain of the present invention contains at least one prebiotic. “Prebiotic” means food substances intended to promote the growth of the probiotic bacterial strain of the present invention in the intestines. The prebiotic may be selected from the group consisting of oligosaccharides and optionally contains fructose, galactose, mannose, soy and/or inulin; and/or dietary fibers.

In some embodiments, the composition that comprises the probiotic bacterial strain of the present invention contains protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

In some embodiments, the administration of the polypeptide of the present invention (or the probiotic bacterial strain that express the protein) is repeated, for example, 2 to 3 times a day, for one day or more and generally for a sustained period of at least 4 days, or even 4 to 15 weeks, with, where appropriate, one or more periods of interruption. In some embodiments, the polypeptide of the present invention is administered simultaneously or sequentially one meal of the subject. In some embodiments, the polypeptide of the present invention is administered prior to the meal of the subject.

As used herein, the term “effective amount” refers to a quantity sufficient of polypeptide of the present invention to achieve the beneficial effect (e.g. treatment of obesity and obesity-related disorders). In the context of the present invention, the amount of a polypeptide of the present invention administered to the subject will depend on the characteristics of the individual, such as general health, age, sex, body weight . . . . The skilled artisan will be able to determine appropriate dosages depending on these and other factors. For example, when the polypeptide of the present invention is administered to the subject in the form a protbiotic, the strain of the present invention shall be able to generate a colony is sufficient to generate a beneficial effect on the subject. If the probiotic bacterial strain is administered in the form of a food product, it typically may comprise between 103 and 10¹² cfu of at the probiotic bacterial strain of the present invention per g of the dry weight of the food composition.

The invention will be further illustrated by the following figures and examples. However, these examples and figures should not be interpreted in any way as limiting the scope of the present invention.

### SEQUENCES

SEQ ID NO: 1:
MMMPAN-X₁-X₂--X₃--X₄-X₅-X₆-X₇-X₈-X₉-X₁₀-X_{11V}-X_{12GG}-X₁₃-
X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-
X₂₉-X₃₀-X₃₁-X₃₂-X₃₃-X₃₄-X₃₅-X₃₆-X₃₇N-X₃₈-X₃₉-X₄₀-X₄₁-X₄₂-
X_{43N}-X₄₄-X₄₅-X₄₆-X₄₇-X₄₈-X₄₉-X₅₀-X₅₁-X₅₂-X₅₃-X₅₄-X₅₅-X₅₆-
X_{57F}-X_{58G}-X₅₉-X₆₀-X₆₁-X₆₂-X₆₃-X₆₄-X₆₅-X₆₆-X₆₇-X₆₈-X₆₉-X₇₀-
X₇₁-X₇₂-X₇₃-X₇₄-X₇₅-X₇₆-X₇₇-X₇₈-X₇₉-X₈₀-X₈₁-X₈₂-X₈₃-X₈₄-X₈₅-
X₈₆-X₈₇-X₈₈-X₈₉-X₉₀-X₉₁-X₉₂-X₉₃-X₉₄-X₉₅-X₉₆-X₉₇-X₉₈-X₉₉-
X₁₀₀-X₁₀₁-X₁₀₂-X₁₀₃-X₁₀₄-X₁₀₅-X₁₀₆-X₁₀₇-X₁₀₈-X₁₀₉-X_{110G}-X₁₁₁-
X₁₁₂-X₁₁₃-X₁₁₄-X₁₁₅ _{Y}-X_{116LG}-X₁₁₇-X₁₁₈-X₁₁₉-X₁₂₀-X₁₂₁-X₁₂₂-
X₁₂₃-X₁₂₄-X₁₂₅-X₁₂₆-X₁₂₇-X₁₂₈-X₁₂₉-X₁₃₀-X₁₃₁-X₁₃₂-X₁₃₃-X₁₃₄-
X₁₃₅-X₁₃₆-X₁₃₇-X₁₃₈-X₁₃₉-X₁₄₀-X₁₄₁-X₁₄₂-X₁₄₃-X₁₄₄-X₁₄₅-X₁₄₆-
X₁₄₇-X₁₄₈-X₁₄₉
wherein:
X1 represents F or Y;
X2 represents S, T;
X3 represents A, V;
X4 represents V, I;
X5 represents S, A, N;
X6 represents E, -;
X7 represents N, -;
X8 represents E, -;
X9 represents M, S, A;
X10 represents T, E;
X11 represents Y, V;
X12 represents M, V, N, Y;
X13 represents S, A;
X14 represents N, D, -;
X15 represents F, L, -;
X16 represents I, F, -;
X17 represents D, T, -;
X18 represents A, I, -;
X19 represents V, L, I;
X20 represents A, V, G;
X21 represents A, D;
X22 represents Y, V, T;
X23 represents L, T;
X24 represents A, P;
X25 represents P, S;
X26 represents A, I;
X27 represents M, W;
X28 represents G, T, N;
X29 represents A, T, L;
X30 represents A, D, E;
X31 represents Q, N, S;
X32 represents W, V;
X33 represents Q, K;
X34 represents N, T, K, R;
X35 represents F, V;
X36 represents H, S, N;
X37 represents K, A, T, S;
X38 represents L, V, I;
X39 represents I, V;
X40 represents T, K;
X41 represents I, L;
X42 represents V, I;
X43 represents G, S;
X44 represents K, S, T;
X45 represents Y, F;
X46 represents V, L, F, T;
X47 represents Q, A, K, S;
X48 represents G, K, S, H;
X49 represents F, Y, T, L;
X50 represents L, T, I, V;
X51 represents D, N, S, K;
X52 represents N, D, R, A;
X53 represents T, V;
X54 represents V, L, I;
X55 represents G, A;
X56 represents A, Q, V, T;
X57 represents V, M, L;
X58 represents S, D, G;
X59 represents T, N, S;
X60 represents W, Y, -,
X61 represents T, V, G,
X62 represents P, T, K, S,
X63 represents G, W, D,
X64 represents D, K, G,
X65 represents G, E, D, V, -,
X66 represents L, V, K, T,
X67 represents T, I, G, L,
X68 represents G, N, F,
X69 represents F, Y, I, G;
X70 represents G, S, E, D;
X71 represents G, -;
X72 represents Q, -;
X73 represents F, -;
X74 represents S, V, -;
X75 represents K, T, -;
X76 represents I, N, -;
X77 represents W, L, -;
X78 represents K, D, E, N;
X79 represents D, K, N, G;
X80 represents N, A, L, S, T;
X81 represents Y, F, I;
X82 represents T, N, G, S;
X83 represents D, T, Q, -;
X84 represents N, -;
X85 represents V, -;
X86 represents T, K, F, -;
X87 represents G, D;
X88 represents E, Y, N, L;
X89 represents S, G, P, W, Y;
X90 represents T, I, N;
X91 represents G, F, E, D, V;
X92 represents A, G, K, D, N;
X93 represents Q, G, N, H, R;
X94 represents K, N, T, L;
X95 represents F, W, T, P;
X96 represents G, R;
X97 represents Y, F, D, T, G;
X98 represents G, Y, D, E, -;
X99 represents A, D;
X100 represents L, V, M, Q;
X101 represents G, N, T;
X102 represents V, A, F;
X103 represents V, L, G;
X104 represents N, -;
X105 represents S, A, K, -;
X106 represents I, G, V, -;
X107 represents L, M, -;
X108 represents N, Q, T, -;
X109 represents V, I, T, -;
X110 represents A, L, -;
X111 represents N, G, I, M, L;
X112 represents L, A;
X113 represents A, S;
X114 represents A, V;
X115 represents I, V, G;
X116 represents N, T;
X117 represents F, S, V, T, M;
X118 represents G, S, A, T, K;
X119 represents T, S, P, D;
X120 represents A, I, T;
X121 represents K, G, A;
X122 represents N, L, V;
X123 represents I, E, T, G, L;
X124 represents V, T, F;
X125 represents G, K, N, A;
X126 represents E, S, D, K;
X127 represents G, T, K;
X128 represents V, T, E, V;
X129 represents Y, L, V, T;
X130 represents K, P, G, N;
X131 represents A, T, FI, S;
X132 represents L, T, N, -;
X133 represents V, G, -;
X134 represents K, Q, -;
X135 represents L, V, -;
X136 represents W, -;
X137 represents G, -;
X138 represents D, -;
X139 represents L, -;
X140 represents P, -;
X141 represents N, -;
X142 represents N, -;
X143 represents G, -;
X144 represents G, -;
X145 represents S, -;
X146 represents G, -;
X147 represents W, -;
X148 represents V, -;
X149 represents G, -;
(-: no amino acid).
SEQ ID NO: 2:
MMMPANYSVIAENEMTYVNGGANFIDAIGAVTAPIWTLDNVKTFNTNI
VTLVGNTFLQSTINRTIGVLFSGNTTWKEVGNIGKNLFGTNVKGNPIEK
NNFGDYAMNALGIAAAVYNLGVAPTKNTVKETEVKFTV
SEQ ID NO: 3:
MMMPANFSAVSENEMTYVMGGSVADYLAPAMGAAQWQNFHKNLITI
VGNKYVQGFLDNTVGAVFSGTWTPGDGLTGFGGQFSKIWKDNYTDN
VTGESTGAQKFGYGALGVVNSILNVAGNLAAIYNLGFGTAKNIVGEGV
YKA
SEQ ID NO: 4:
MMMPANFSAVSENEMTYVMGGSVADYLAPAMGAAQWQNFHKNLITI
VGNKYVQGFLDNTVGAMFSGTWTPGDGLTGFGGQFSTIWKKNYTDN
VTDESTGAQKFGYGALGVVNSILNVAGNLAAIYNLGFGTAKNIVGEGV
YKA
SEQ ID NO: 5:
MMMPANFSAVAENEMTYVVGGSLVDVLAPAMTTANWQNVSANVIKI
VGNSFLAKYTNDVLAQLFDGNYVPGDVIGYSVKNLDKAYNKGYGTFG
GNWGFAVGALNAGMQILGGLSAIYTLGSSSIGLETKSGTLPTL
SEQ ID NO: 6:
MMMPANFTAVNSEVVYGGADLFTILADTTAPIWNAANVKKFNTNLIT
LISNSFFKKTVSNTLGVMFGGNWGKDGDKIFGEEGSINQNVFGLWNDD
HTTRTDDMTFGNKVMQVLGMAAVGYTLGTTDAKVGFNDGVYGING
KL
SEQ ID NO: 7:
MMMPANFSAVNAEVVYGGAVADYLPSAWTAESVKRFNSNIITLVSNS
FTSHLLKATLGTMFSGSWGSDGVTLFGDNGTFSGLYNVNRLPGGEAQT
FGNKIMTTLGLASVVYTLGMKDAAVLTAKKVTNSNGQVWGDLPNNG
GSGWVG
SEQ ID NO: 8:
VTLVGNTFLQSTINRTIGVL
SEQ ID NO: 9:
IWTLDNVKTFNTNIVTLVGNTFLQSTINRTIGVLFSGNTTWKE
SEQ ID NO: 10:
ITIVGNKYVQGFLDNTVGAV
SEQ ID NO: 11:
ITIVGNKYVQGFLDNTVGAM
SEQ ID NO: 12:
IVGNSFLAKYTNDVLAQL
SEQ ID NO: 13:
ITLISNSFFKKTVSNTLGVM
SEQ ID NO: 14:
ITLVSNSFTSHLLKATLGTM
SEQ ID NO: 15:
VTIIGNSYVSKLVGATLGVM
SEQ ID NO: 16:
ITIIGNSYVSKVLGATLGVM
SEQ ID NO: 17:
IVGNKYVQGFLDNTVGAV
SEQ ID NO: 18:
AMGAAQWQNFHKNLITIVGNKYVQGFLDNTVGAVFSGTWTPGD
SEQ ID NO: 19:
AMGAAQWQNFHKNLITIVGNKYVQGFLDNTVGAMFSGTWTPGD
SEQ ID NO: 20:
AMTTANWQNVSANVIKIVGNSFLAKYTNDVLAQLFDGNYVPGD
SEQ ID NO: 21:
IWNAANVKKFNTNLITLISNSFFKKTVSNTLGVMFGGNWGKDG
SEQ ID NO: 22:
AWTAESVKRFNSNIITLVSNSFTSHLLKATLGTMFSGSWGSDG
SEQ ID NO: 23:
IWTTANVKTFNTNLVTIIGNSYVSKLVGATLGVMFGGNWGGDG
SEQ ID NO: 24:
VWGAANVKTFNTNLITIIGNSYVSKVLGATLGVMFSGAWGTKD
SEQ ID NO: 25:
AMGAAQWQNFHKNLVTIVGNKYVQGFLDNTVGAVFSGTWTPGV
SEQ ID NO: 26:
FSGNTTWKEVGNIGKNLFGTNVKGNPIEKNNFGDYAMNALGIA
SEQ ID NO: 27:
GNTFLQSTINRTIGVL
SEQ ID NO: 28:
VGNTFLQSTINRTIGVL
SEQ ID NO: 29:
LVGNTFLQSTINRTIGVL
SEQ ID NO: 30:
TLVGNTFLQSTINRTIGVL
SEQ ID NO: 31:
VTLVGNTFLQSTINRTIGVL
SEQ ID NO: 32:
IWTLDNVKTFNTNIVTLVGNTFLQSTINRTIGVLFSGNTTWK
SEQ ID NO: 33:
MMMPANYSVIAENEMTYVNGGANFIDAIGAVTAPIWTLDNVKTFNTNI
VTLVGNTFLQSTINRTIGVLFSGNTTWKEVGNIGKNLFGTNVKGNPIEK
NN
SEQ ID NO: 34:
NFIDAIGAVTAPIWTLDNVKTFNTNIVTLVGNTFLQSTINRTIGVLFSG
NTTWKEVGNIGKNLFGTNVKGNPIEKNNFGDYAMNALGIAAAVYNLGVA
PTKNTVKETEVKFTV
SEQ ID NO: 35:
NTFLQSTINRTIGVL
SEQ ID NO: 36:
AAVYNLGVAPTKNTVKETEVKFTV
SEQ ID NO: 37:
NYSVIAENEMTYVNGGANFIDAIGAVTAPIWTLDNVKTFNTNIVTLV
SEQ ID NO: 38:
KGNTFLQSTINRTIGVL
SEQ ID NO: 39:
VKGNPIEKNNFGDYAMNALGIAAAVYNLGVAPTKNTVKETEVKFTV

### FIGURES

FIG. 1 demonstrated that L-MAM treatment prevented animals from development of High-Fat Diet-induced obesity. Absolute body weight (g) during intervention time. L-MAM treated mice significantly gained less weight throughout the gavage as compared to HFD-fed control experiment. a: P<0.05 versus NCD; b: P<0.05 versus HFD60%; c: P<0.05 versus HFD60%+L-empty.

FIG. 2: Magnetic resonance imaging whole-body composition analysis. The difference in body weight between L-MAM-treated animals on a HFD and the remaining animals is largely due to a significant reduction in the percentage of fat mass and a significant increase in the percentage of lean mass. a: P<0.05 versus NCD; b: P<0.05 versus HFD60%; c: P<0.05 versus HFD60%+L-empty.

FIG. 3: (A) Plasma glucose (mg dl⁻¹) profile and (B) the mean area under the curve (AUC-30-120 min) measured during an oral tolerance test (OGTT). Oral glucose tolerance testing (OGTT) revealed that chronic administration of L-MAM increased glucose tolerance compared to HFD-mice or mice with chronic administration of L-empty. a: P<0.05 versus NCD; b: P<0.05 versus HFD60%; c: P<0.05 versus HFD60%+L-empty.

FIG. 4: Relative weight (as % of body weight at time of termination) of subcutaneous adipose tissue (mWAT); mesenteric adipose tissue and perigonadal adipose tissue. All adipose tissues weights were lower in L-MAM-treated mice compared to HFD or L-empty-treated mice. a: P<0.05 versus NCD; b: P<0.05 versus HFD60%; c: P<0.05 versus HFD60%+L-empty.

### EXAMPLE

Methods

Animals and Diets.

C57BL/6 7 week old mice were housed under a 12:12 hour light-dark cycle. All mice were fed the control diet for a period of 1 week to acclimatize them. After 1 week the mice were assigned to one of 4 experimental conditions for the following 12 weeks. The control group (NCD) remained on the control diet while the high fat diet group was fed a high fat diet (60% kcal fat). Visual health and activity checks were performed daily by trained animal care staff and cages changed twice a week. Animals were weighed once a week for the duration of the study.

Plasmid Construction.

MAM encoding plasmid (pIL-MAM) was created by a fusion between pIL253 cut with PstI (Fermentas) and pCMV including DNA of MAM (see above) cut with Sbf1. Empty equivalent (pIL-EMPTY) was created using the same method, but with fusion of pIL253 empty pCMV. pIL-MAM and pIL-EMPTY were transformed in Lactococcus lactis MG1363 as described by Langella et al. (1993). L. lactis strains were thereafter grown on M17 medium.

Bacterial Strains and Growth Conditions.

L. lactis MG1363 containing pIL-EMPTY (L-empty) plasmid and L. lactis MG1363 containing pIL-MAM plasmid (L-MAM) were grown in M17 medium (Difco) supplemented with 1% glucose and erythromycin (10 μg/mL) at 30° C. without agitation overnight. The next day, the cultures were diluted 1/20 in M17 medium and grown up at 30° C. without agitation. Based on our knowledge, at OD=1 the bacteria concentration is around 5×10⁸ CFU/mL. Mice were gavaged with 5×10⁹ CFU/mouse. For all gavages, aliquots 10× concentrate were previously prepared as described and frozen at −80° C. To use, aliquots were gradually thaw on ice bath to preserve all structures and diluted with PBS.

Glucose Tolerance Tests.

Age-matched mice were fasted for 6 hr prior to testing. The blood glucose baseline was determined using Roche ACCU-CHEK Aviva blood glucose monitor and strips. Mice were gavaged with a bolus of glucose dissolved in 0.9% saline. The amount of glucose was dependent upon body weight. Blood glucose was monitored every 30 min for 2 hours.

MIR.

The percentage of body fat was determined using an EchoMRI-900 Body Composition Analyzer. The percentage body fat and body lean mass were calculated.

Treatment of Mice

All mice were fed a standard laboratory chow diet, except the HFD 60% groups.

Four groups of mice were formed:
the first control group of mice (NCD) was administrated drinking water
the second group of mice (HFD60%) was fed a high fat diet for 12 weeks
the third group of mice (HFD60%+L-empty) was fed a high fat diet 60% for 12 weeks and treated with L-empty (gavage with 5×10⁹ CFU per mouse each day), starting the day of HFD and until the end of the protocol
the fourth group of mice (HFD 60%+L-MAM) was fed a high fat diet 60% for 12 weeks and treated with L-MAM (gavage each day), starting the day of HFD and until the end of the protocol.

Results

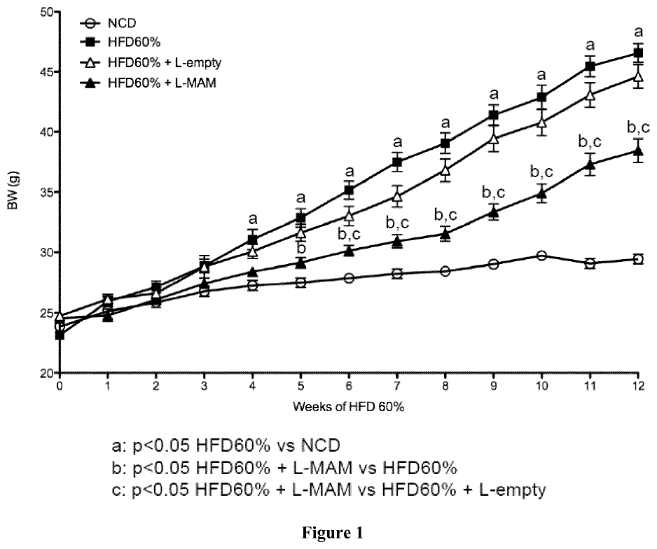

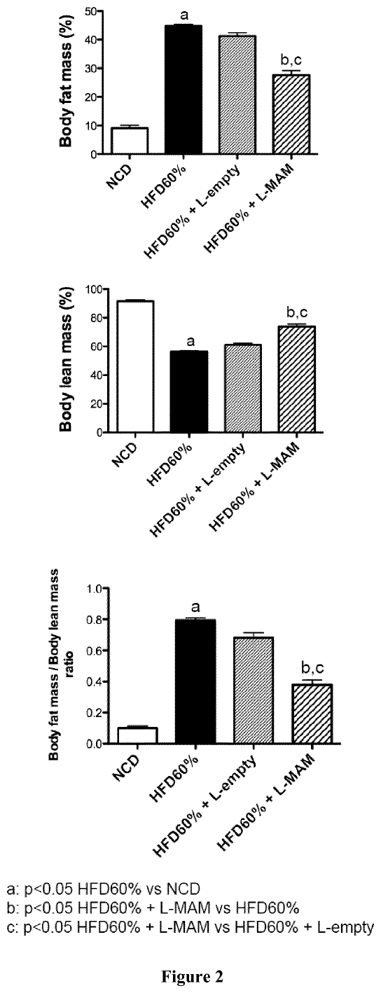

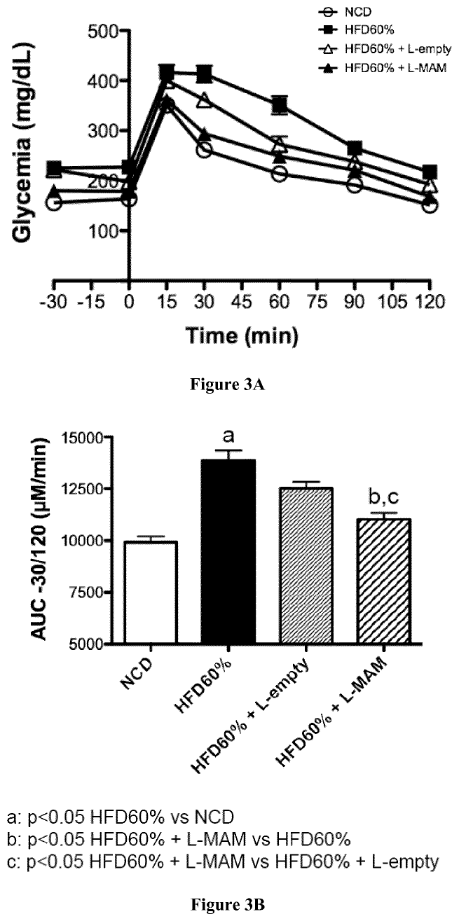

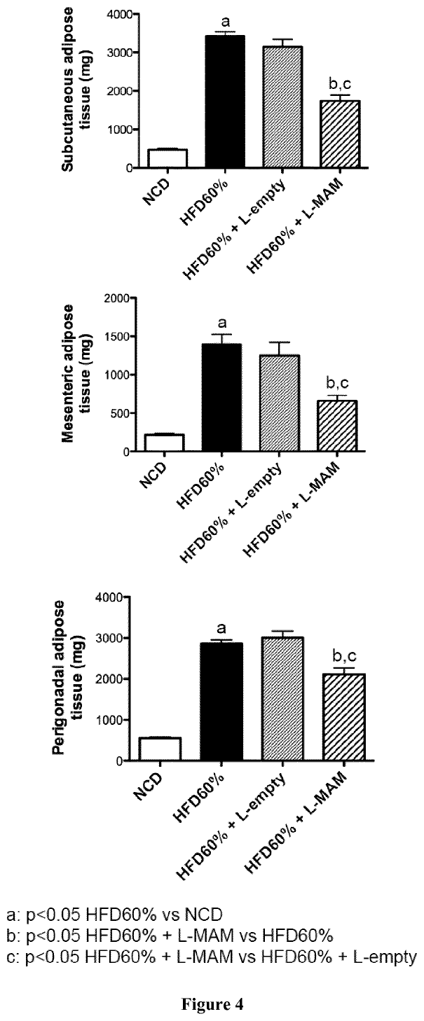

The results are depicted in FIGS. 1-4. In particular, FIG. 1 shows that L-MAM treatment prevented animals from development of High-Fat Diet-induced obesity. L-MAM treated mice significantly gained less weight throughout the gavage as compared to HFD-fed control experiment. The difference in body weight between L-MAM-treated animals on a HFD and the remaining animals is largely due to a significant reduction in the percentage of fat mass and a significant increase in the percentage of lean mass (FIG. 2). Oral glucose tolerance testing (OGTT) revealed that chronic administration of L-MAM increased glucose tolerance compared to HFD-mice or mice with chronic administration of L-empty (FIG. 3). Finally, all adipose tissues weights (of subcutaneous adipose tissue (mWAT); mesenteric adipose tissue and perigonadal adipose tissue) were lower in L-MAM-treated mice compared to HFD or L-empty-treated mice relative weight (FIG. 4).

### REFERENCES

Throughout this application, various references describe the state of the art to which this invention pertains. The disclosures of these references are hereby incorporated by reference into the present disclosure.

1. A method of treating obesity or an obesity-related disease in a subject in need thereof comprising administering to the subject a therapeutically effective amount of:
a polypeptide having the amino acid sequence as set forth in SEQ ID NO:1 or a fragment thereof,
a nucleic acid molecule encoding for a polypeptide having the amino acid sequence as set forth in SEQ ID NO:1 or a fragment thereof,
a vector comprising a nucleic acid molecule encoding for a polypeptide having the amino acid sequence as set forth in SEQ ID NO:1 or a fragment thereof, and/or
a host cell transformed with a nucleic acid molecule encoding for a polypeptide having the amino acid sequence as set forth in SEQ ID NO:1 or a fragment thereof,
wherein the obesity-related disease is selected from the group consisting of bulimia, diabetes, hypertension, elevated plasma insulin concentrations and insulin resistance, dyslipidemia, hyperlipidemia, breast, prostate, endometrial cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack, and osteoarthritis,
or from the group consisting of metabolic syndrome, also known as syndrome X, insulin resistance syndrome, type II diabetes, impaired fasting glucose, impaired glucose tolerance, hypercholesterolemia, hyperuricaemia, and left ventricular hypertrophy, or is a non-alcoholic fatty liver disease,
and is in particular a non-alcoholic fatty liver disease.

2. The method of claim 1 wherein the obesity-related disease is a non-alcoholic fatty liver disease that is nonalcoholic steatohepatitis (NASH).

3. The method of claim 1 wherein the obesity-related disease is insulin resistance.

4. The method of claim 1 comprising administering to the subject a therapeutically effective amount of:
polypeptide having an amino acid sequence selected in the group comprising SEQ ID NO:2, SEQ ID NO:3, SEQ ID NO:4, SEQ ID NO:5, SEQ ID NO:6 and SEQ ID NO:7, or a fragment thereof
a nucleic acid molecule encoding for a polypeptide having an amino acid sequence selected in the group comprising SEQ ID NO:2, SEQ ID NO:3, SEQ ID NO:4, SEQ ID NO:5, SEQ ID NO:6 and SEQ ID NO:7, or a fragment thereof
a vector comprising a nucleic acid molecule encoding for a polypeptide having an amino acid sequence selected in the group comprising SEQ ID NO:2, SEQ ID NO:3, SEQ ID NO:4, SEQ ID NO:5, SEQ ID NO:6 and SEQ ID NO:7, or a fragment thereof
a host cell transformed with a nucleic acid molecule encoding for a polypeptide having an amino acid sequence selected in the group comprising SEQ ID NO:2, SEQ ID NO:3, SEQ ID NO:4, SEQ ID NO:5, SEQ ID NO:6 and SEQ ID NO:7. Or a fragment thereof.

5. The method of claim 1 wherein the fragment consists in 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50; consecutive amino acid residues in SEQ ID NO:1, SEQ ID NO:2, SEQ ID NO:3, SEQ ID NO:4, SEQ ID NO:5, SEQ ID NO:6 or SEQ ID NO:7, in particular has an amino acid sequence selected in the group consisting of SEQ ID NO: 8-39, and more particularly has an amino acid sequence selected in the group consisting of SEQ ID NO: 8, SEQ ID NO: 26 and SEQ ID NO: 36.

6. The method of claim 1 wherein the host cell is a probiotic bacterial strain.

7. The method of claim 6 wherein the probiotic bacterial strain is viable or not viable.

8. The method of claim 6 wherein the probiotic bacterial stain is selected from food grade bacteria.

9. The method of claim 6 wherein the probiotic bacterial strain is Lactococcus lactis.

10. The method of claim 6 wherein the probiotic bacterial strain is administered to the subject in the form of a food composition.

11. The method of claim 10 wherein the food composition that comprises the probiotic bacterial strain comprises an amount of dietary fibers.

12. The method of claim 10 wherein the food composition that comprises the probiotic bacterial strain contains at least one prebiotic.
